(19) United States
(12) Patent Application Publication
(10) Pub. No.: US 2014/0221772 A1
(43) Pub. Date: Aug. 7, 2014

(54) BODY SURFACE SENSORS

(71) Applicants: Lior Wolloch, Ramat Gan (IL); Yoni Epstein, Ramat Hasharon (IL); Adi Eldar, Kiryat Ono (IL); Michael Nagler, Tel Aviv (IL); Konstantin Goulitski, Holon (IL); Nir Eden, Haifa (IL); Haim Melman, Kfar Sava (IL)

(72) Inventors: Lior Wolloch, Ramat Gan (IL); Yoni Epstein, Ramat Hasharon (IL); Adi Eldar, Kiryat Ono (IL); Michael Nagler, Tel Aviv (IL); Konstantin Goulitski, Holon (IL); Nir Eden, Haifa (IL); Haim Melman, Kfar Sava (IL)

(73) Assignee: DEEP BREEZE LTD., Or Akiva (IL)

(21) Appl. No.: 14/117,040

(22) PCT Filed: May 16, 2012
(86) PCT No.: PCT/IB12/52465

§ 371 Date: Nov. 12, 2013

Related U.S. Application Data
(60) Provisional application No. 61/457,719, filed on May 18, 2011.

(51) Int. Cl. A61B 5/0408; A61B 5/00; A61B 5/0416; A61B 7/00; A61B 5/0205

(52) U.S. Cl. CPC A61B 5/04087; A61B 7/003; A61B 5/0205; A61B 5/0416; A61B 5/4878
USPC 600/301; 600/586

(57) ABSTRACT

A device for sensing lung sounds, comprising: a piezoelectric sensor comprising an electrical conductive plate attached to a piezoelectric material, said sensor encased in a body structure; a first electric wire connected to the piezoelectric material on the opposite side of said plate; a second electric wire connected to said plate; a connector connected to the other ends of said first and second electric wires; and an adhesive layer connected to the surface of said plate on the side opposite to the piezoelectric material, said adhesive layer facing away from said plate; said device adapted to provide electrical signals representing vibrations present on the surface of a object when it is attached to said object surface with said adhesive layer; said electrical signals resulting from vibrations on the object surface, wherein stress applied on the piezoelectric material generates electrical voltage-difference on both sides of the piezoelectric material, creating voltage build-up on said first and second electric wires.

### CROSS-REFERENCE TO RELATED PATENT APPLICATIONS

The present application is a national phase application of International Patent Application No. PCT/IB2012/052465 (PCT Publication No. WO2012/156930), filed May 16, 2012, and entitled “BODY SURFACE SENSORS”, which claims priority to U.S. Provisional Patent Application 61/457,719, filed May 18, 2011, and entitled “BODY SURFACE SENSORS”, the disclosures of which are incorporated herein by reference in their entireties.

### TECHNICAL FIELD

The present invention deals with sensors for continuously monitoring vital signs, and more particularly with disposable sensor for continuously monitoring lung sounds, electrical activity of the heart and other vital signs.

### BACKGROUND OF THE INVENTION

There are disposable stethoscopes such as Veridian 05-13503 Single Patient Use Disposable Stethoscope (http://www.amazon.com/gp/product/B003UYOUPiezoelectric?tag=theshoclo-20).

There are electronic stethoscopes such as 3M Littmann Electronic Stethoscope—3100 Mode (http://solutions.3m.com/wps/portal/3M/en_US/Littmann_{—}3100_{—}3200/stethoscope/)

None of the above is suitable for continuous monitoring of lung sounds.

There is also no solution for low cost continuous lung sound sensors that can be used for one patient and be disposed after such usage.

### SUMMARY

Provide low cost lung sound sensors suitable for continuous monitoring of lung sounds.

Provide low cost lung sound sensors suitable for continuous monitoring of lung sounds that also support other vital signs monitoring.

Particularly provide low cost lung sound sensors suitable for continuous monitoring of lung sounds that also support ECG sensing.

Particularly provide low cost lung sound sensors suitable for continuous monitoring of lung sounds that also support temperature sensing.

Particularly provide low cost lung sound sensors suitable for continuous monitoring of lung sounds that also function as a sound generator for broadcasting sound waves into the body tissue and recording of the transmitted sounds using same sensors acting as sound sensors.

Providing combinations of the above functionality in a single sensor while minimizing the area that such a sensor occupies on the patient's body.

Providing combinations of the above functionality in a single sensor while minimizing the number of electronic wires required to operate such a senor.

### DETAILED DESCRIPTION OF THE INVENTION

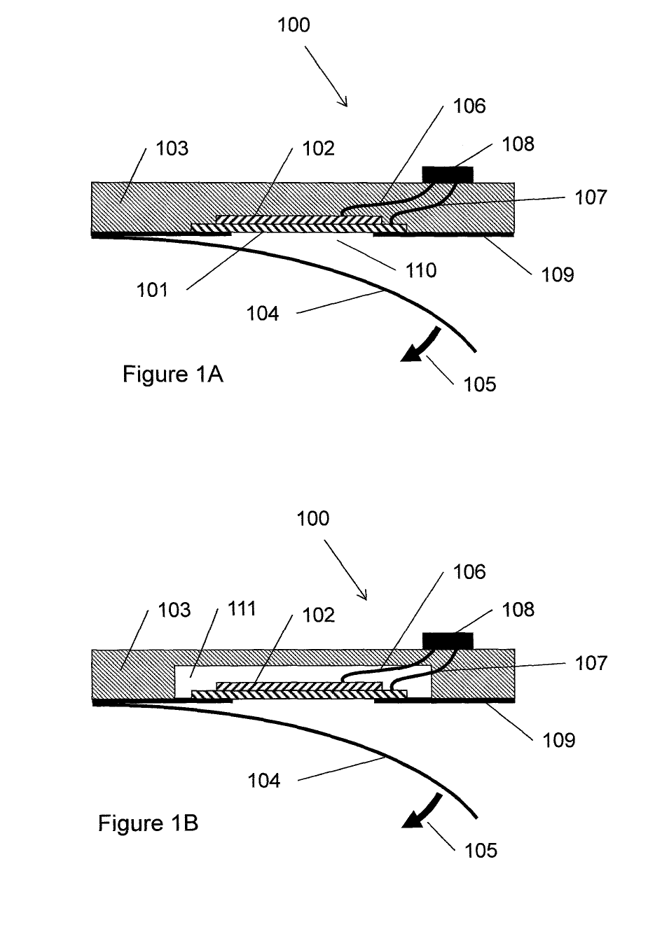

Reference is made now to FIG. 1A that represents one preferred embodiment of the present invention.

In this invention, the term “Piezoelectric device” means a layer of piezoelectric material of any kind, in any form of design. In the specific example of the current invention, a Piezoelectric device is represented by the example of an assembly of a layer of piezoelectric ceramics, assembled with a layer of a conductive metal substrate such as Model OBO-TE32211-26 available from OBO Pro.2 Inc., Pa-Te City, Tauyuan, Taiwan.

It would be appreciated that the invention is not limited to this specific Piezoelectric device.

Sensor 100 is constructed from a Piezoelectric device. Such a Piezoelectric sensor is constructed from electrical conductive thin disk 101 attached to a Piezoelectric ceramics 102. When stress is applied on the Piezoelectric device, electrical voltage-difference is generated on both sides of the Piezoelectric ceramics. This results in voltage build-up on wires 106 and 107 that are connected to external connector 108. This characteristic provides for using such a Piezoelectric device to record vibrations such as present on the body surface as a result from the breathing sounds of a patient.

The low cost lung sounds sensor 100 is further constructed of a body of material 103 that supports the structure described hereinabove. By selecting suitable material for 103 one can also provides isolation from environment noises to support recording of more pure sound coming from the measured surface. A variety of polyurethane materials can serve such purposes.

It would be appreciated that body of material 103 is not required for the basic function of device 100 and device 100 can function without it. Body martial 103 is provided here as an improved embodiment of the invention and does not limit the scope of the invention.

Layer 109 carries an adhesive layer to enable the attachment of device 100 to the surface of a patient. The adhesive would typically be of the types used for ECG stickers such as Medi-Trace 230 ECG Conductive Adhesive Electrodes available from BP Medical Supplies, Brooklyn, N.Y., USA (http://www.bpmedicalsupplies.com/product.sc?productId=657).

When used for lung sound monitoring, layer 109 can cover the whole bottom surface of device 100. In this preferred embodiment, layer 109 has an opening 110 that exposes conductive metal 101, thereby enabling galvanic contact between conductive metal 101 and the skin of the patient. This is provided as an example for a sensor that can also support measuring electrical signals from the body of the patient and it does not limit the scope of the current invention to the configuration of this example.

Liner 104 is typically made of a polymer and serves to protect adhesive layer 109 from dust or occidental contact. When ready for attachment of device 100 to the skin of the patient, liner 104 is peeled-off as shown by arrow 105, in the same way that such a liner is used with common ECG adhesive electrodes.

The device of FIG. 1A can be attached to the skin of a person and connected through connector 108 and, preferably, a coax or dual lead shielded cable, to any reading device having suitable analog and digital electronics suitable to record the signal out of this device. Such reading devices are available such as VRIxp from Deep Breeze Ltd., Or Akiva, Israel but are also well established art involving pre-amplifier circuits, analog to digital conversion of the signal and storing in a storage device for further processing by a computer.

FIG. 1B provides enhancement to the structure of FIG. 1A in the form of volume 111 that can be used as an air gap to separate the Piezoelectric element from relatively hard and heavy body structure 103. If heavy and hard material is selected for body structure 103 to provide better isolation from environment noise, a direct contact of the Piezoelectric element might drastically affect its' response for the vibrations coming from the patient's skin.

Volume 111, being an air cell or being filled with a relatively soft material can provide the Piezoelectric device with the stress-free environment, allowing for proper sensing of vibrations coming from the patient's skin.

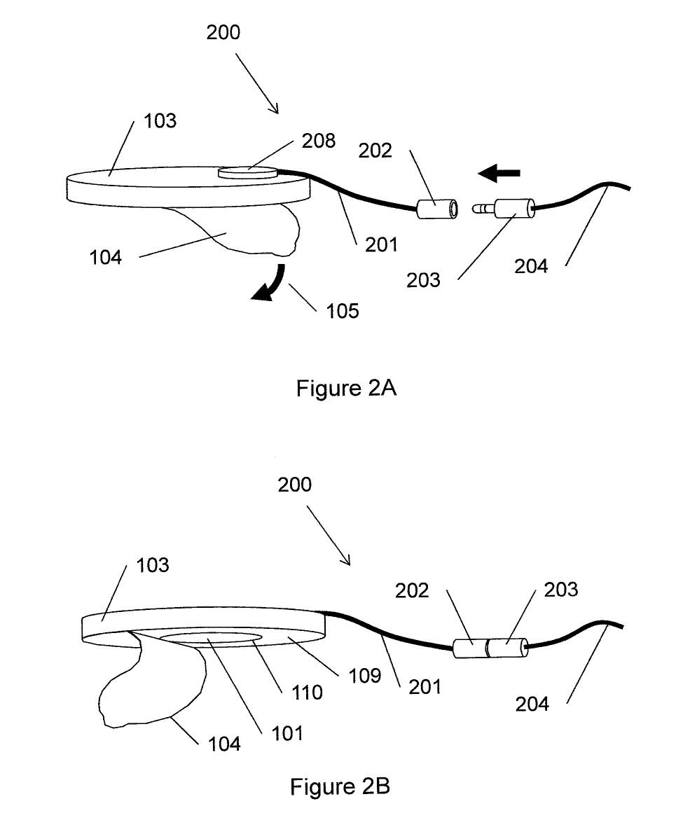

Reference is made now to FIG. 2 which provides a 3D view of device 100 of FIG. 1.

FIG. 2A is a top view of device 200. This preferred embodiment is different from the embodiment of FIG. 1 by element 208 that differs from socket 108 of FIG. 1 by providing an anchor structure for cable 201 having an on-cable socket 202. Cable 204 with plug 203 can connect to socket 202 as shown in FIG. 2B to transfer the signal produced by device 200 to a suitable electronic device.

FIG. 2B presents the device of FIG. 2A with the adhesive side 109 visible.

Opening 110 in the adhesive layer is shown and conductive metal surface 101 of the Piezoelectric device is also shown here.

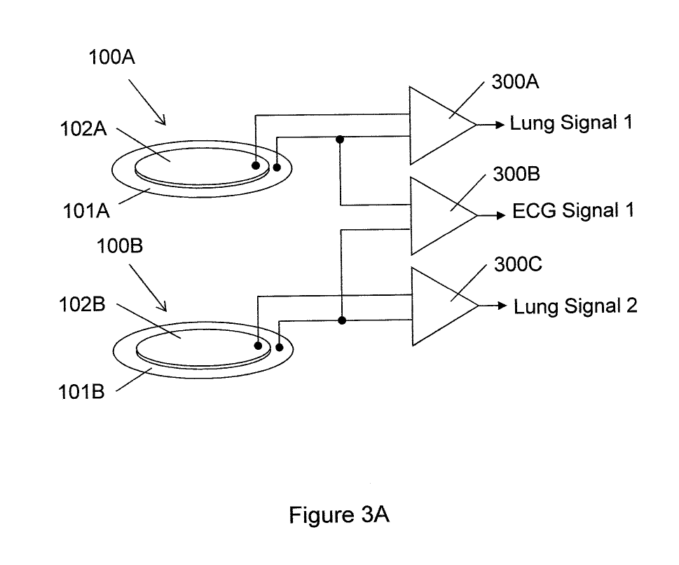

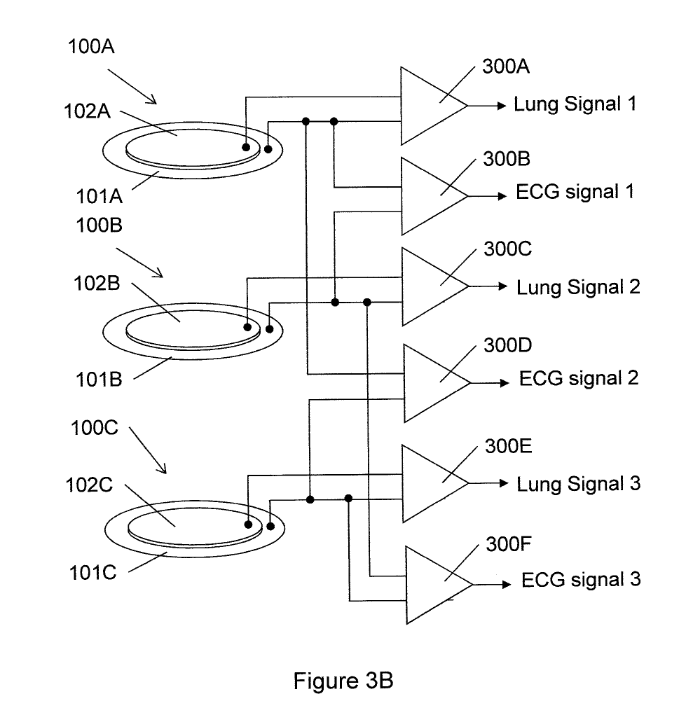

Reference is made now to FIG. 3A. FIG. 3A provides an example of how two Piezoelectric devices can be connected to electronic circuit to support both the signals for lung sounds and skin electrical signals such as ECG.

For clarity, most of the construction elements of device 100 (or 200) were removed from this drawing, presenting only the Piezoelectric devices 100A with its two components: Piezoelectric ceramics layer 102A and conductive metal layer 101A and Piezoelectric 100B with its two components: Piezoelectric ceramics layer 102B and conductive metal layer 101B.

300A, 300B and 300C are 3 differential amplifiers.

The input side of differential amplifier 300A is connected the upper surface of Piezoelectric ceramics 102A and to conductive metal 101A. When Piezoelectric device 100A is exposed to vibrations (such as skin vibrations resulting from lung sounds) the voltage difference on the two input wires of differential amplifier 300A results in Lung Signal 1 useful as an electrical representation of the lung sound signals.

The input side of differential amplifier 300C is connected the upper surface of Piezoelectric ceramics 102B and to conductive metal 101B. When Piezoelectric device 100B is exposed to vibrations (such as skin vibrations resulting from lung sounds) the voltage difference on the two input wires of differential amplifier 300C results in Lung Signal 2 useful as an electrical representation of the lung sound signals.

The input side of differential amplifier 300B is connected to the conductive metal 101A of Piezoelectric device 100A and to the conductive metal 101B of Piezoelectric device 100B. When Piezoelectric devices 100A and 100B are exposed to voltage difference on the skin of the patient, the voltage difference on the on the two input wires of differential amplifier 300B results in ECG Signal 1 useful as an electrical representation of the electrical voltage difference of electrodes 101A and 101B. Just as required for ECG.

It would be appreciated that measurement of skin voltage differences is useful for a variety of applications and ECG is provided as a support of one example only, without limiting the scope of the present invention.

FIG. 3B is an example of how the concept of FIG. 3A can be expanded to utilize additional sensors using the same method.

Here Piezoelectric device 100C is added.

Also differential amplifiers 300D, 300E and 300F are added.

With the same method of FIG. 3A, differential amplifier 300E provides additional Lung Signal 3.

Additional differential amplifier 300D provides ECG Signal 2 for electrodes 101A and 101C.

Additional differential amplifier 300F provides ECG Signal 3 for electrodes 101B and 101C.

It will be appreciated that this method can be repeated in the same way to support any number of sensing devices of the current invention, including, but not limited to, 12 leads ECG reading with 12 such sensing devices.

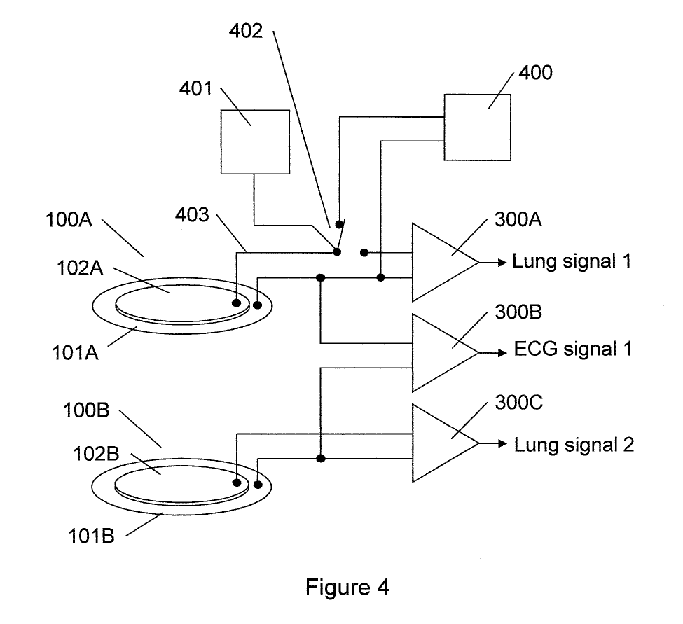

Reference is made now to FIG. 4 providing another embodiment of the present invention.

The configuration of FIG. 4 is based on the configuration of FIG. 3A except that lead 403 of Piezoelectric device 100A can now be switched between differential amplifier 300A to provide the sensing function of Lung Signal 1 as described in reference to FIG. 3A or switched to signal generator 400 as shown in FIG. 4. The switching operation is made through control circuit 401 that represents any switch controlling circuit that can be derived by any electrical signal.

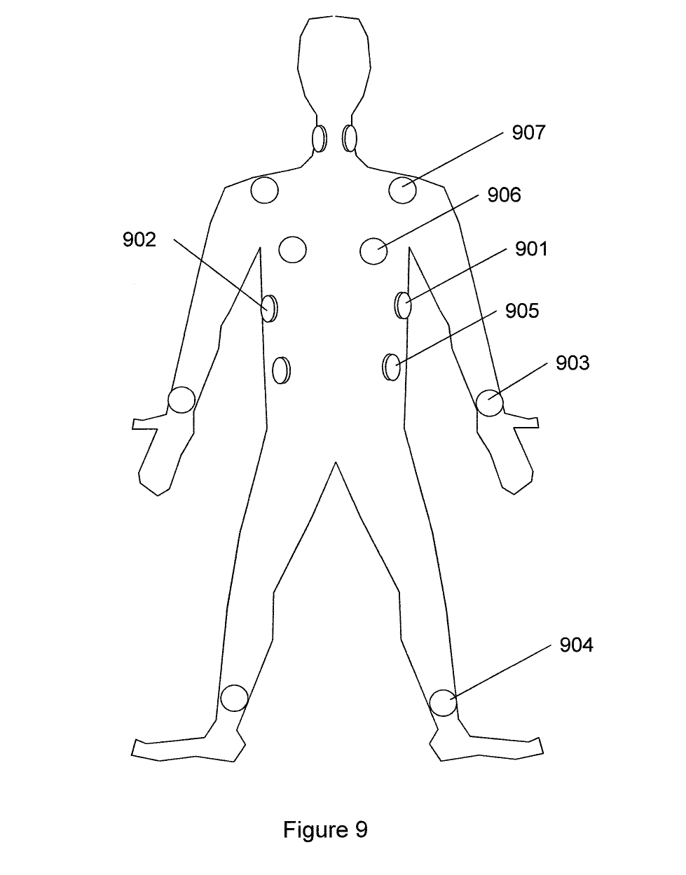

In this configuration of FIG. 4, device 100A can by attached to the patient skin on one side of the torso and device 100B can be attached to another side of the torso, as shown by numerical references 901 and 902 of FIG. 9.

In this configuration Piezoelectric device 100A can be used alternatively to inject vibrations into the patient body (as shown in FIG. 4) or as a regular lung sounds sensor (as it functions when connected to differential amplifier 300A).

In this position of switch 402, signal generator 400 is used to generate sounds.

Such typical sounds might be a series of different sinusoidal frequencies and given amplitudes. Each of the sinusoidal frequencies is sensed by Piezoelectric device 100B after traveling through the body tissue. The amplitude of the sinusoidal signals at the position of Piezoelectric device 100B depends on the content of the tissue separating Piezoelectric device 100A from Piezoelectric device 100B and the frequency of the sinusoidal signal. This can be utilized, for example, to estimate amount of water in the lungs of the patient.

FIG. 4 therefore, demonstrate the 3-function capability of the invention, to provide in one simple and low cost sensor measurements of lung sounds, ECG and tissue content analysis. In more generalized words, the three functions enabled by this embodiment of the invention is sensing skin vibrations, skin galvanic potential differences and injecting vibrations into the body of the patient.

It would also be appreciated that the configuration of FIG. 3 and FIG. 4 can be used with the Piezoelectric device as is, stripped from the various packaging mechanics of the current invention, using only 2-sided tape to attaché the Piezoelectric device to the skin of the patient. Although this is not a referred embodiment of the current invention, the invention is not limited to packaged Piezoelectric devices.

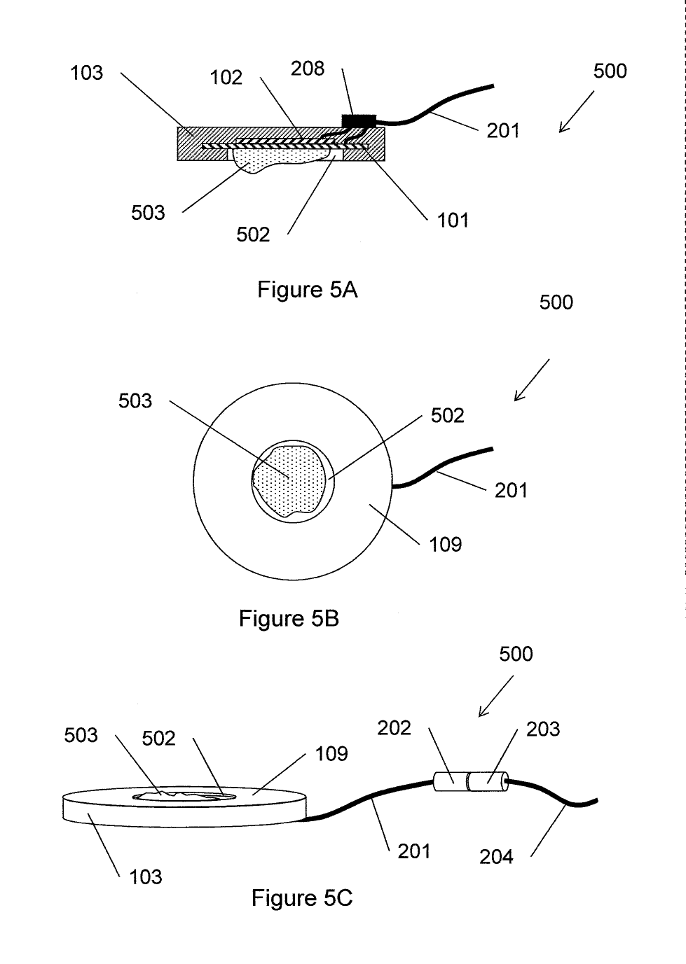

Reference is made now to FIG. 5. This embodiment of the invention is a variation of device 100 of FIG. 1A. In this embodiment, the Piezoelectric device is positioned in a deeper location in the body material 103 as shown in FIG. 5A. Open cavity 502 just below conductive metal 101 of the Piezoelectric device provides for placement of conductive gel 503 such as CG04 Saline Base Signa Gel available from Bio-Medical Instruments, Inc., Warren, Mich. USA.

Usage of such a gel improves the electrical contact for the galvanic signals of the present invention. The gel also provides improved interface to transfer the skin vibration to the Piezoelectric device when irregular skin surface might deteriorate such an interface quality.

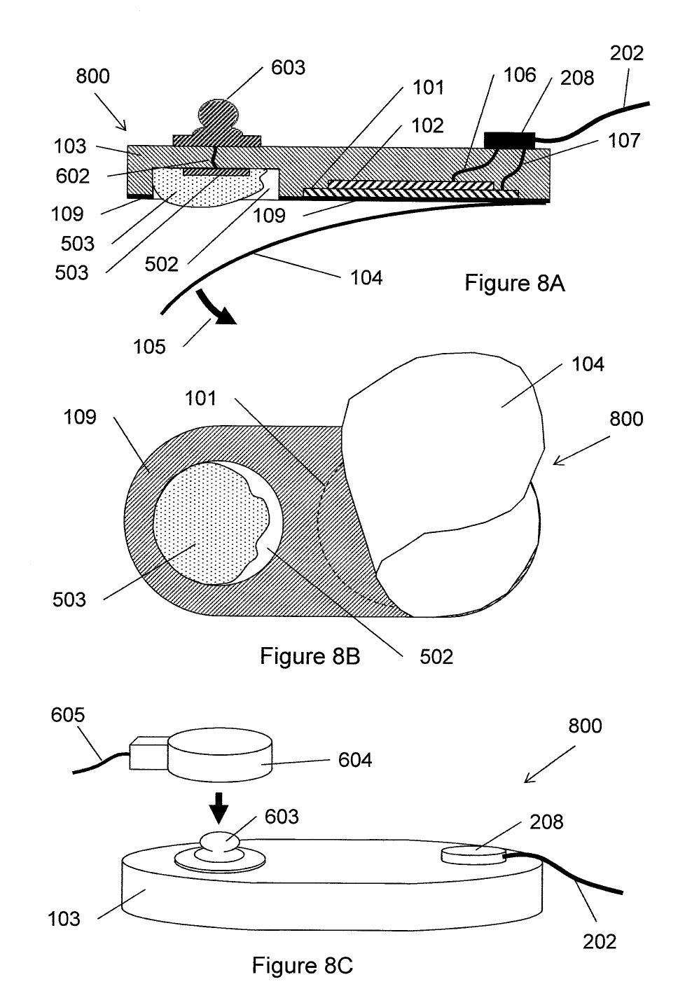

The gel may be included in device 500 covered with liner 104 to protect the device until it is used as shown in FIG. 8A and FIG. 8B.

FIG. 5B and FIG. 5C provide additional views of device 500 of FIG. 5.

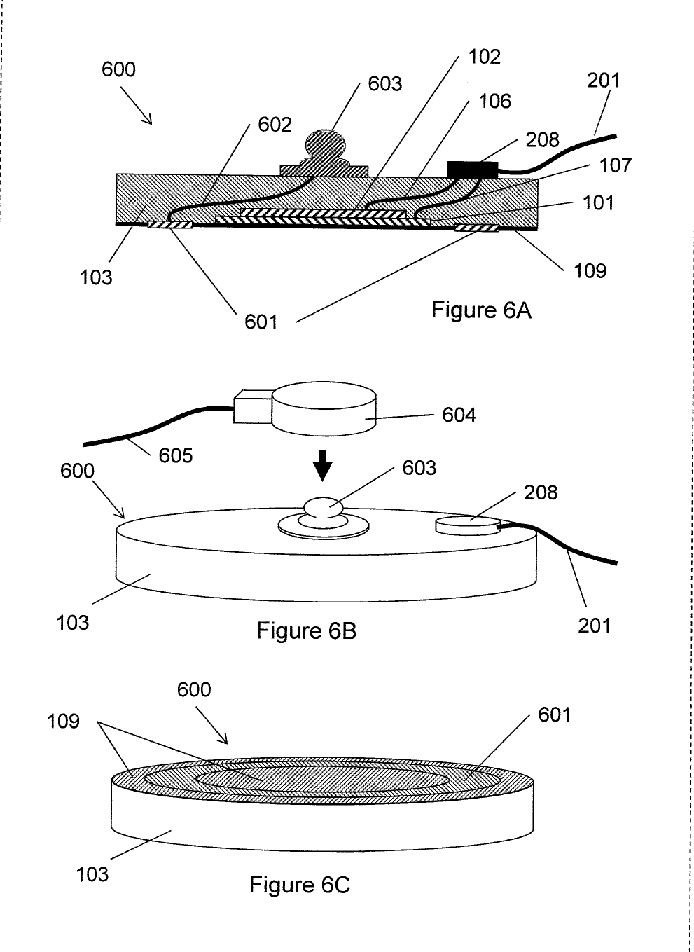

Reference is made now to FIG. 6 which represents yet another preferred embodiment of the invention where the galvanic skin contact is separated from the Piezoelectric device.

In FIG. 6A, a cross section of device 600, a conductive ring 601 is attached to the skin side of device 600. Lead 602 connects between conductive ring 601 and snap-button 603 which, in this example, is a standard ECG snap-button suitable for many models of ECG leads such as Welch Allyn ECG Lead Wires for Atlas Monitor available from Welch Allyn Inc., Skaneateles Falls, N.Y., USA.

Piezoelectric device and its contacts are the same as in FIG. 1A and 2A.

This configuration supports the usage of this structure with common ECG devices such as the ECG Atlas Monitor of Welch Allyn and does not require the specifically designed circuits of FIG. 3A and FIG. 3B. This is achieved at the cost of additional lead 605 with the snap-head 604 of FIG. 6B.

FIG. 6C provides a 3D view of the skin side of device 600 of FIG. 6.

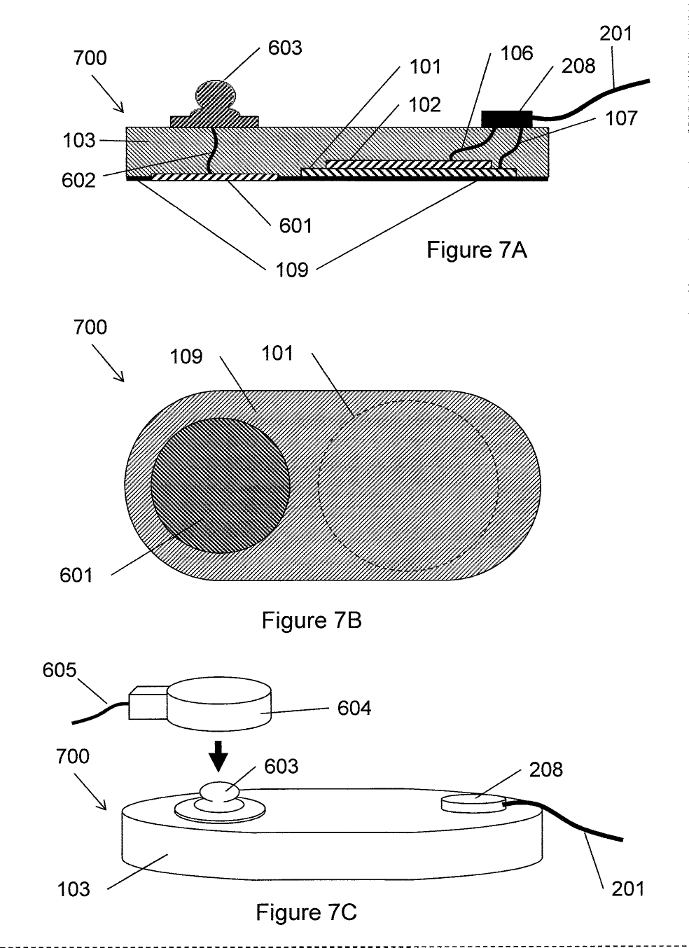

Reference is made now to FIG. 7 which is yet another embodiment that is a variation of the embodiment of FIG. 6.

Unlike device 600 of FIG. 6 where Piezoelectric device and the galvanic contact device are concentric, in the embodiment of FIG. 7 the two elements are arranged side-by-side.

FIG. 7A provides a cross section of device 700 with the conductive layer 601 positioned to the left of the Piezoelectric device and connected to the ECG snap-button 603 with lead 602 to provide the required galvanic contact to the ECG lead.

It would be appreciated that the 3 components 601, 602 and 603 can be constructed out of one conductive piece that provides both sides of the contacts: 601 and 603.

FIG. 7B provides a view of the skin side of device 700. On the left side, conductive layer 601 is shown and is available for contact with the skin of the patient.

Adhesive layer 109 covers the complete area except for the opening required for conductive layer 601 to provide for the necessary skin contact.

The location of Piezoelectric device conductive metal 101 is shown by a dashed line and is covered by adhesive layer 109.

FIG. 7C provides a 3D illustration of device 700, including the illustration of an ECG snap-head 604 and lead 605.

Reference is made now to FIG. 8 which represents yet another preferred embodiment of the current invention.

FIG. 8 provides a combination of device 700 of FIG. 7 and conductive gel 503 of FIG. 5.

In this embodiment, conductive layer 503 is mounted in cavity 502, thus providing for conductive gel 503. The function of conductive gel 503 in this example is only to enhance galvanic contact and it has no function in reference to the Piezoelectric device as in FIG. 5.

In FIG. 8A and FIG. 8B liner 104 is also, shown partially peeled off.

FIG. 9 is an illustration of optional positioning of the devices of the current invention over the body of a patient.

Locations such as 901 and 902 are particularly useful for measurement of spectral transmission of vibration signals across the tissue that includes the lungs as explained hereinabove.

Locations such as 903 and 904 are particularly useful in reference to ECG measurements.

Locations such as 905, 906 and 907 are useful both for lung sounds signals and ECG signals.

It would be appreciated that the locations of the devices of the present invention can vary without limitation per the application for which they are used and that the examples above are provided only as such without limiting the scope of the invention.

It would also be appreciated that the different combinations design features of the device of the current invention are provided as examples of preferred embodiment and do not limit the scope of the invention.

The scope of the invention is specified only by the claims.

1. A device for sensing lung sounds, comprising:
a piezoelectric sensor comprising an electrical conductive plate attached to a piezoelectric material, said sensor encased in a body structure;
a first electric wire connected to the piezoelectric material on the opposite side of said plate;
a second electric wire connected to said plate;
a connector connected to the other ends of said first and second electric wires; and
an adhesive layer connected to the surface of said plate on the side opposite to the piezoelectric material, said adhesive layer facing away from said plate;
said device adapted to provide electrical signals representing vibrations present on the surface of a object when it is attached to said object surface with said adhesive layer;
said electrical signals resulting from vibrations on the object surface, wherein stress applied on the piezoelectric material generates electrical voltage-difference on both sides of the piezoelectric material, creating voltage build-up on said first and second electric wires.

2. The device of claim 1 wherein said object is a body of a person and said vibrations are those caused by breathing.

3. The device of claim 1, wherein said body structure comprises sound isolation material.

4. The device of claim 3, wherein said isolation material is polyurethane.

5. The device of claim 1, wherein said sensor is mounted as the bottom surface of said body structure.

6. The device of claim 5, wherein said adhesive layer comprises an opening that exposes said conductive plate, thereby enabling galvanic contact between the conductive disk and the surface of an object, said device adapted to additionally measure electrical signals from the surface of the object.

7. The device of claim 1, additionally comprising a protective liner attached to the external surface of said adhesive layer.

8. The device of claim 6, wherein said sensor is mounted inside said body structure, additionally comprising an open cavity exposing said conductive plate, said cavity adapted to holding conductive gel adapted to improve the electrical contact for the galvanic signals and to providing improved interface of the object surface vibrations to the piezoelectric sensor.

9. The device of claim 1, wherein said connector is connected by a third electric wire to a reading device adapted to recording the signals from said device.

10. The device of claim 9, wherein said third electric wire comprises one of a coax cable and a dual lead shielded cable.

11. The device of claim 9, wherein said third electric wire comprises an on-cable socket adapted to connect to a plug, said plug adapted to transfer the signal produced by device to a reading device.

12. The device of claim 1, wherein said body structure comprises a volume adapted to separate the piezoelectric sensor from the body structure.

13. The device of claim 12, wherein said volume comprises one of air and soft material.

14. The device of claim 1, additionally comprising a galvanic surface contact, said galvanic surface contact electrically connected to an ECG snap-button mounted on top of said device.

15. The device of claim 14, wherein said piezoelectric sensor and said galvanic contact are mounted concentrically.

16. The device of claim 14, wherein said piezoelectric sensor and said galvanic contact are mounted side-by-side.

17. The device of claim 16, wherein said galvanic contact is mounted inside said body structure, additionally comprising an open cavity exposing said galvanic contact, said cavity adapted to holding conductive gel adapted to improve the electrical contact between the galvanic contact and the surface of the object.

18. A system comprising:
a plurality of devices for sensing lung sounds, each device comprising:
a piezoelectric sensor comprising an electrical conductive plate attached to a piezoelectric material, said sensor encased in a body structure;
a first electric wire connected to the piezoelectric sensor;
a second electric wire connected to the electrical conductive plate;
an external connector connected to the other ends of said first and second electric wires; and
an adhesive layer connected to the surface of said electrical conductive plate facing away from the piezoelectric material,
and
an electronic circuit comprising a plurality of first amplifiers adapted to provide lung signals,
wherein each said first amplifiers is connected on its input side to said first and second electric wires of one of said plurality of devices,
and wherein each said first amplifiers is adapted to provide a signal representing the voltage difference on said first and second electric wires resulting from the vibrations of the surface of the object.

19. The system of claim 18, wherein each said adhesive layers comprises an opening that exposes the conductive plate, thereby enabling galvanic contact between the conductive plate and the surface of an object,
additionally comprising a plurality of second amplifiers,
wherein each said second amplifiers is connected on its input side to two of said second electric wires, each from a different one of said plurality of devices,
and wherein each said second amplifiers is adapted to provide a signal representing the electrical voltage difference on the two input wires,
said system adapted to provide an electrical representation of surface vibration signals and surface electric potential differences signals.

20. The system of claim 18, additionally comprising at least one signal generator controlled by a control circuit, said control circuit adapted to switch at least one electric wire of one said devices between said the amplifier and said signal generator,
said system adapted to alternately sense surface vibrations of an object and inject sounds into an object.

21. The system of claim 20, wherein said injected sounds comprise a series of different sinusoidal frequencies and given amplitudes.

22. The system of claim 20, comprising a first device connected to a signal generator and a second device, said first and second devices adapted to be attached to two locations of an object surface, wherein a sound injected through said first device is sensed by said second device after traveling through the object, the amplitude of the signal generated by said second device depends on the content of said object.

23. The system of claim 22 wherein said system adapted to provide an electrical representation of the lung sound signals and to analyze tissue content.

24. The system of claim 23, wherein said tissue content comprises water content in the lungs.

25. The system of claim 19, additionally comprising at least one signal generator controlled by a control circuit, said control circuit adapted to switch at least one electric wire of one said devices between said first amplifier connected to it and said signal generator,
said system adapted to alternately use all sensors to record lung sounds or use part of the sensors to inject sounds into an object while the other part of the sensors used to sense vibrations from the surface of said object and, additionally, also adapted to sense surface electric potential differences.

26. The system of claim 25, wherein said injected sounds comprise a series of different sinusoidal frequencies and given amplitudes.

27. The system of claim 25, comprising a first device connected to a signal generator and a second device, said first and second devices adapted to be attached to two sides of a patient's torso, wherein a sound injected into said first device is sensed by said second device after traveling through the body tissue, the amplitude of said signal depending on the content of said body tissue,
said system adapted to provide an electrical representation of the lung sound signals and ECG signals and to analyze tissue content.

28. The system of claim 27, wherein said tissue content comprises water content in the lung.
